(19) United States
(12) Patent Application Publication
(10) Pub. No.: US 2004/0231010 A1
(43) Pub. Date: Nov. 18, 2004

(54) Lysozyme transgenic ungulates

(76) Inventors: James D. Murray, Davis, CA (US); Gary B. Anderson, Davis, CA (US); Elizabeth A. Maga, Sacramento, CA (US); Sushma Pati, Fremont, CA (US); David A. Zarling, Fremont, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/753,266

(22) Filed: Jan. 7, 2004

Related U.S. Application Data
(60) Provisional application No. 60/480,356, filed on Jun. 20, 2003.
(60) Provisional application No. 60/439,150, filed on Jan. 9, 2003.

(51) Int. Cl. A01K 67/027

(52) U.S. Cl. 800/14; 800/15

(57) ABSTRACT

The present invention provides transgenic ungulates that include a transgene that encodes lysozyme, and further has an attenuated or non-functional β-lactoglobulin allele. The invention further provides methods for producing such animals. The invention further provides methods of producing a food product, such as milk, or a milk product, using a subject transgenic ungulate, as well as food products harvested from a subject transgenic ungulate.

### CROSS-REFERENCE

This application claims the benefit of U.S. Provisional Patent Application No. 60/439,150 filed Jan. 9, 2003, and of U.S. Provisional Patent Application No. 60/480,356 filed Jun. 20, 2003, which applications are incorporated herein by reference in their entirety.

### FIELD OF THE INVENTION

The present invention is in the field of transgenic non-human animals, and milk products produced by such animals.

### BACKGROUND OF THE INVENTION

Cheese yield determines profits for the dairy industry since it represents the amount of product made from a given amount of milk. Milk composition and quality influence cheese making properties such as coagulation time (rennet clotting time), gel strength and cheese yield.

Cheese is generally made by acidifying the milk and setting the milk with a clotting agent, such as rennet, or by developing acidity to the isoelectric point of the protein. The set milk is cut and whey is separated from the resulting curd. The curd may be pressed to provide a cheese block. The whey, which contains significant amounts of whey protein, is generally further processed for protein and fat recovery. Curing typically takes place over an extended period of time (often several months or longer) under controlled conditions. Process cheese-type products can be prepared from such conventional cheeses by grinding and then heating the ground cheeses with emulsifying salt.

There have been many efforts to provide simplified processes for making cheese or cheese-type products, thereby increasing production efficiency. For example, elimination of the whey drainage step, and other modifications to the standard process have been described.

In spite of the numerous attempts and the clear advantages that a simplified process would provide, there exists a need in the art for improved methods of cheese production. The present invention addresses this need by providing lysozyme transgenic ungulates that have a non-functional β-lactoglobulin allele. Milk produced by a subject transgenic ungulate exhibits reduced rennet clotting time, and has enhanced bacteriostatic properties. Cheese products produced from milk of a subject transgenic ungulate also have increased gel strength. These and other advantages are discussed in detail below.

Literature

U.S. Pat. Nos. 5,891,698, 6,140,552, 6,268,487, 6,140,552, 5,741,957, 6,013,857, 6,118,045, 5,994,616, 5,907,080, 5,750,176, 5,892,070, 6,204,431; WO 97/05771; WO 01/00855; Gutierrez-Adan et al. (1999) J. Dairy Res. 66:289-294; Maga et al. (1998) J. Food Prot. 61:52-56; Maga et al. (1995) J. Dairy Sci. 78:2645-2652; Maga and Murray (1995) Bio/Technology 13:1452-1457; Maga et al. (1994) Transgenic Res. 3:36-42.

### SUMMARY OF THE INVENTION

The present invention provides transgenic ungulates that include a transgene that encodes lysozyme, and further has an attenuated or non-functional β-lactoglobulin allele. The invention further provides methods for producing such animals. The invention further provides methods of producing a food product, such as milk, or a milk product, using a subject transgenic ungulate, as well as food products harvested from a subject transgenic ungulate.

### FEATURES OF THE INVENTION

The present invention features a transgenic ungulate that includes a transgene encoding lysozyme and a non-functional β-lactoglobulin allele. In some embodiments, the transgenic ungulate is female, the lysozyme-encoding transgene is expressed in mammary gland cells of the ungulate, the milk produced by the transgenic ungulate has a level of lysozyme that is at least about 10% higher than the level of lysozyme in milk of a non-transgenic ungulate of the same species, and the milk has a level of β-lactoglobulin that is at least about 10% lower than the level of β-lactoglobulin in milk of a non-transgenic ungulate of the same species. In some of these embodiments, the transgene includes a coding sequence for lysozyme operably linked to a mammary specific promoter. In other embodiments, the ungulate is male.

In some embodiments, the ungulate is a goat. In some embodiments, the transgene is chromosomally integrated. In some embodiments, the ungulate is heterozygous for the transgene and heterozygous for the non-functional β-lactoglobulin allele. In other embodiments, the ungulate is homozygous for the transgene and homozygous for the non-functional β-lactoglobulin allele. In some embodiments, the transgene encodes human lysozyme.

The present invention further features an isolated fertilized egg, where the egg is isolated from a transgenic ungulate that includes a transgene encoding lysozyme and a non-functional β-lactoglobulin allele. The invention further provides a composition including such a fertilized egg; and a cryoprotective agent.

The present invention further features a method of producing a food product, where the method involves harvesting a food product from a transgenic ungulate of the present invention. The present invention further features a method of producing a food product, the method involving processing a food product harvested from a transgenic ungulate of the present invention.

The present invention further features a method of producing cheese, the method involving: adding rennet to milk harvested from a transgenic ungulate of the present invention; allowing curd formation to occur; and producing cheese from the curds.

The present invention further features a milk product produced by a transgenic ungulate of the present invention, where the milk product has a level of lysozyme that is at least about 10% higher than the level of lysozyme in milk of a non-transgenic ungulate of the same species, and where the milk product has a level of β-lactoglobulin that is at least about 10% lower than the level of P-lactoglobulin in milk of a non-transgenic ungulate of the same species. In some embodiments, the milk product has reduced rennet clotting time compared to a milk product of a non-transgenic animal of the same species. In some embodiments, the milk product has enhanced bacteriostatic properties compared to a milk product of a non-transgenic animal of the same species. In some embodiments, the milk product has increased shelf life compared to the shelf life of the same milk product made from milk of a non-transgenic animal of the same species.

The present invention further features a processed milk produced from milk of a transgenic ungulate of the present invention. In some embodiments, the processed milk product is cheese. In some of these embodiments, the cheese has increased gel strength compared to cheese made from milk of a non-transgenic ungulate of the same species.

Definitions

The term “transgene” is used herein to describe genetic material which has been or is about to be artificially inserted into the genome of a non-human animal, and particularly into a cell of a living non-human mammal.

The term “transformation” refers to a permanent or transient genetic change induced in a cell following the incorporation of new DNA (i.e. DNA exogenous to the cell). Where the cell is a mammalian cell, a permanent genetic change is generally achieved by introduction of the DNA into the genome of the cell.

The term “ES cell” as used herein refers to pluripotent embryonic stem cells and to such pluripotent cells in the very early stages of embryonic development, including but not limited to cells in the blastocyst stage of development.

The term “construct” refers to a recombinant nucleic acid, generally recombinant DNA, that has been generated for the purpose of the expression of a specific nucleotide sequence(s), or is to be used in the construction of other recombinant nucleotide sequences.

The term “operably linked” refers to a functional connection between a DNA sequence and a regulatory sequence(s), e.g., a DNA sequence and a regulatory sequence(s) are connected in such a way as to permit gene expression when the appropriate molecules (e.g., transcriptional activator proteins) are bound to the regulatory sequence(s).

The term “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein.

The term “genomic sequence” refers to a sequence having non-contiguous open reading frames, where introns interrupt the protein coding regions. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kbp or smaller; and substantially free of flanking chromosomal sequence.

### DETAILED DESCRIPTION OF THE INVENTION

The present invention provides transgenic ungulates that include a transgene that encodes lysozyme, which transgenic ungulates also include an attenuated or non- functional β-lactoglobulin allele, and methods for producing such animals. The subject transgenic animals are useful for producing milk. The milk produced by a subject transgenic animal has a higher level of lysozyme, as well as a lower level of β-lactoglobulin, than milk of a non-transgenic animal of the same species. The elevated level of lysozyme and the reduced level of β-lactoglobulin confer various advantages on the milk. For example, because of the elevated level of lysozyme and the reduced level of β-lactoglobulin, the milk has a reduced rennet clotting time, and is therefore advantageous for production of cheese, as cheese yield is increased. Furthermore, the cheese produced by such milk has increased gel strength. The lysozyme is also bacteriostatic toward a variety of undesirable bacteria, and therefore the presence of increased levels of lysozyme in the milk increases food safety. Furthermore, the bacteriostatic properties of the lysozyme reduce the incidence of mammary gland infections (mastitis) in the milk-producing transgenic ungulate. Because β-lactoglobulin is an allergen to human infants, a reduction in β-lactoglobulin levels reduces the allergenicity of milk produced by a subject transgenic animal.

The subject invention further provides methods of producing a milk product from a subject transgenic ungulate, by harvesting the milk from the subject transgenic ungulate. The subject invention also provides methods of making food products from the milk of a subject transgenic ungulate. The subject invention further provides milk harvested from a subject transgenic ungulate, and food products made with such milk.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a transgene” includes a plurality of such transgenes and reference to “the milk product” includes reference to one or more milk products and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### TRANSGENIC NON-HUMAN ANIMALS AND METHODS FOR THEIR PRODUCTION

The present invention provides transgenic ungulates that include a lysozyme transgene and that has an attenuated or non-functional β-lactoglobulin allele. A lysozyme transgene includes a nucleotide sequence that encodes lysozyme. The lysozyme coding sequence is any coding sequence that, when transcribed and translated, provides for production of enzymatically active lysozyme. The lysozyme coding sequence is operably linked to one or more control elements that provide at least for transcription of the coding sequence. In many embodiments, e.g., where the transgenic ungulate is a female, the lysozyme coding sequence is operably linked to a mammary gland-specific promoter such that the encoded lysozyme is produced in the mammary gland. Suitable coding sequences and promoters are described in more detail below.

A subject transgenic ungulate has an attenuated or non-functional β-lactoglobulin allele. A β-lactoglobulin allele can be rendered non-functional in a variety of ways. In some embodiments, the lysozyme transgene replaces an endogenous β-lactoglobulin allele. In other embodiments, one or more mutations are introduced into a β-lactoglobulin allele, which mutation(s) render it non-functional. A β-lactoglobulin allele can be attenuated (e.g., where the level of transcription and/or translation of the allele or an mRNA copy of the coding region of the allele, is reduced, such that the level of β-lactoglobulin polypeptide produced is reduced, compared to the wild-type, non-mutated allele) in a variety of ways. One or more mutations are introduced into the β-lactoglobulin allele, which mutation(s) render the allele attenuated. For example, a mutation is introduced into the promoter region or other transcriptional control region of the β-lactoglobulin allele, such that the level of transcription of the allele is reduced.

In some embodiments, a subject transgenic ungulate has one attenuated or non-functional β-lactoglobulin allele. In other embodiments, a subject transgenic ungulate has two attenuated or non-functional β-lactoglobulin alleles.

The transgenic ungulates of the present invention are milk-producing agricultural ungulates, including, but not limited to, goats, sheep, and cows. The present invention provides both female and male lysozyme transgenic animals. A subject transgenic animal is heterozygous or homozygous for the lysozyme transgene. A subject transgenic animal is heterozygous or homozygous for the attenuated or non-functional β-lactoglobulin allele. Males are useful for breeding with female ungulates of the same species to produce transgenic offspring that are homozygous or heterozygous for the lysozyme transgene and that are heterozygous or homozygous for the attenuated or non-functional β-lactoglobulin allele. Females are useful both for production of milk with increased levels of lysozyme and decreased levels of β-lactoglobulin, and for production of transgenic offspring.

A subject female transgenic ungulate produces milk that has a level of enzymatically active lysozyme that is at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 4-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, at least about 1200-fold, at least about 1500-fold, at least about 1700-fold, at least about 2000-fold, at least about 2500-fold, or at least about 3000-fold, or more, higher than the level of enzymatically active lysozyme in the milk of a female non-transgenic animal of the same species. For example, the level of enzymatically active lysozyme in the milk of a subject transgenic ungulate is from about 0.10 mg/ml to about 2.0 mg/ml, from about 0.2 mg/ml to about 1.0 mg/ml, or from about 0.4 mg/ml to about 0.8 mg/ml. In some embodiments, the level of enzymatically active lysozyme in the milk of a subject transgenic ungulate is greater than 2.0 mg/ml, e.g., from about 2.0 mg/ml to about 7.5 mg/ml, e.g., from about 2.0 mg/ml to about 2.5 mg/ml, from about 2.5 mg/ml to about 3.0 mg/ml, from about 3.0 mg/ml to about 3.5 mg/ml, from about 3.5 mg/ml to about 4.0 mg/ml, from about 4.0 mg/ml to about 5.0 mg/ml, from about 5.0 mg/ml to about 6.0 mg/ml, from about 6.0 mg/ml to about 7.0 mg/ml, or from about 7.0 mg/ml to about 7.5 mg/ml.

Methods of measuring the level of lysozyme polypeptide, and methods of measuring the level of enzymatically active lysozyme, in milk are known in the art, and any known method can be used. Suitable methods for measuring the level of lysozyme polypeptide in milk include, but are not limited to, Western blotting (e.g., applying a sample to a polyacrylamide gel, electrophoresing the sample, transferring proteins in the gel to a membrane, and detecting lysozyme on the membrane using an antibody specific for lysozyme), followed by densitometric scanning; an enzyme linked immunosorbent assay (ELISA) using antibody specific for lysozyme; and the like. Suitable methods for measuring enzymatic activity of lysozyme include, but are not limited to, an assay for clearing of bacteria incorporated into a gel; bacterial clearing in solution (using a turbidometric assay); and the like. In many assays, lysozyme activity is measured by clearing of Micrococcus lysodeikticus.

A subject female transgenic ungulate produces milk that has a level of β-lactoglobulin that is at least about 5%, at least about 10%, at least about 15%, at least about 20%, at least about 25%, at least about 30%, at least about 35%, at least about 40%, at least about 45%, or at least about 50%, at least about 60%, at least about 70%, at least about 80%, or at least about 90% less than the level of β-lactoglobulin in the milk of a non-transgenic animal of the same species.

Any known method can be used for determining the level of β-lactoglobulin in the milk of a subject transgenic animal. For example, an immunological assay, employing an antibody specific for β-lactoglobulin, can be used, e.g., in an ELISA assay, a Western blot assay, and the like.

A subject transgenic ungulate produces milk that is bacteriostatic, particularly toward Gram-positive bacteria. Milk from a subject transgenic ungulate is at least about 5%, at least about 10%, at least about 15%, at least about 20%, at least about 25%, at least about 30%, at least about 35%, at least about 40%, at least about 45%, or at least about 50%, at least about 60%, at least about 70%, at least about 80%, at least about 90%, at least about 2-fold, at least about 4-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, at least about 1200-fold, at least about 1500-fold, at least about 1700-fold, at least about 2000-fold, at least about 2500-fold, or at least about 3000-fold, or more, bacteriostatic than milk from a non-transgenic ungulate of the same species. In particular, the milk of a subject ungulate exhibits bacteriostatic activity toward mastitis-causing bacteria, and toward bacteria that cause food spoilage. Such bacteria include, but are not limited to, Escherichia coli; various staphylococcal species, including, e.g., Staphylococcus areus; Pseudomonas species, including, e.g., Pseudomonas fragi, P. fluorescens, P. aeruginosa; streptococcal species, including e.g., Streptococcus cremoris, S. agalactiae, S. uberis; Lactococcus species, including, e.g., L. lactis; Lactobacillus species; and the like.

The level of bacteriostatic activity of a sample of milk is readily determined using well-known assays. For example, a bacterial suspension is added to a milk sample, and the milk sample is incubated at 37° C. for various periods of time, after which times dilutions of the cultures are plated onto agar containing bacterial growth medium, and the number of bacteria per culture determined. The level of bacteriostatic activity of the milk sample is inversely related to the number of bacteria.

The milk, or a product made from milk of a subject transgenic ungulate has increased shelf life compared to the shelf life of the same milk product made from milk of a non-transgenic animal of the same species. For example, the shelf life of milk or a product made from the milk of a subject transgenic ungulate has a shelf life that is at least about 10%, at least about 15%, at least about 20%, at least about 25%, at least about 30%, at least about 35%, at least about 40%, at least about 50%, at least about 60%, at least about 70%, at least about 80%, at least about 90%, at least about 2-fold, at least about 3-fold, at least about 4-fold, or at least about 5-fold or more greater than the shelf life of the milk or the same milk product made from milk of a non-transgenic animal of the same species.

The milk of a subject transgenic ungulate has reduced rennet clotting time, compared to the milk of a non-transgenic ungulate of the same species. The rennet clotting time is the time required for curd formation to occur. A reduction in rennet clotting time provides for enhanced cheese production. The rennet clotting time of milk of a subject transgenic ungulate is reduced by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the rennet clotting time of milk of a non-transgenic ungulate of the same species.

Rennet clotting time is determined using any known method, including the method described in the Example. Methods for determining rennet clotting time are known in the art. See, e.g., Maga et al. (1995) J. Dairy Sci. 78:2645-2652. Rennet clotting times of two or more milk samples are compared under the same temperature conditions, and curd formation is measured at a given time after addition of rennet.

Cheese produced from the milk of a subject transgenic ungulate has increased gel strength compared to cheese made by the same process from milk of a non-transgenic ungulate of the same species. The gel strength of cheese produced from the milk of a subject transgenic ungulate is at least about 5%, at least about 10%, at least about 15%, at least about 20%, at least about 25%, at least about 30%, at least about 35%, at least about 40%, at least about 45%, at least about 50%, or more, greater than the gel strength of cheese made by the same process from milk of a non-transgenic ungulate of the same species. Increased gel strength is measured using any known method, e.g., the method described in the Example.

Methods of Making a Subject Transgenic Animal

The invention provides methods of generating a subject transgenic animal. The method generally involves introducing a lysozyme transgene into a pluripotent or totipotent cell such that the transgene is integrated into the genome of the cell, and transferring the cell into an oviduct of a synchronized recipient female of the same species as the cell.

In some embodiments, the lysozyme transgene includes sequences that provide for homologous recombination with an endogenous β-lactoglobulin allele. In these embodiments, the lysozyme transgene includes 5′ and 3′ flanking sequences that are homologous to sequences in the 5′ and 3′ regions of an endogenous β-lactoglobulin gene. The 5′ and 3′ flanking sequences each independently include from about 10 to about 15, from about 15 to about 20, from about 20 to about 30, from about 30 to about 40, from about 40 to about 50, or from about 50 to about 100contiguous nucleotides, or more, that share from about 90% to about 100% nucleotide sequence identity to a sequence of contiguous nucleotides of the same length in the β-lactoglobulin allele. As a result of homologous recombination, all or a portion of the β-lactoglobulin allele is replaced with the lysozyme transgene.

In other embodiments, one or more mutations are introduced into the β-lactoglobulin gene such that the allele is either rendered non-functional or is attenuated. For example, one of the following mutations are generated: (1) all or part of the β-lactoglobulin allele can be deleted, using homologous recombination with a sequence other than a lysozyme transgene; (2) one or more mutations are introduced into the β-lactoglobulin promoter such that the activity of the β-lactoglobulin promoter is reduced; (3) one or more mutations are introduced that affect an mRNA splice site such that the mRNA is not correctly spliced; (4) a frameshift mutation is introduced into the β-lactoglobulin gene such that a truncated protein is produced; (5) a mutation is introduced such that a stop codon is introduced into the coding region, such that a truncated protein is produced; and the like.

Sequences of all or part of β-lactoglobulin alleles of various ungulates are known and publicly available. For example, the sequence of the β-lactoglobulin gene of Capra hircus (goat) is found under GenBank accession no. Z33881; the sequence of the β-lactoglobulin gene of Ovis aries (sheep) is found under GenBank accession number X12817; the sequence of the β-lactoglobulin gene of Bos taurus (cow) is found under GenBank accession number X14710. Other β-lactoglobulin gene sequences (including promoter regions, 5′ flanking regions, 3′ flanking regions, introns, exons, and cDNA sequences) are found in public databases.

Suitable 5′ and 3′ flanking sequences of an endogenous β-lactoglobulin allele to be used in a DNA construct include regions surrounding the start codon of the β-lactoglobulin gene. For example, a cDNA encoding lysozyme (or other suitable protein) is flanked by β-lactoglobulin sequences corresponding to at least about 25, at least about 50, at least about 75, or at least about 100 bp on either side of the start codon. In this fashion, when homologous recombination occurs, the start codon of the endogenous β-lactoglobulin gene will be replaced with the cDNA of the replacement transgene. This will result in the expression of the replacement transgene under control of the β-lactoglobulin gene, and a non-functional β-lactoglobulin protein.

As discussed above, in some embodiments, the lysozyme transgene replaces all or part of an endogenous β-lactoglobulin allele. In other embodiments, an β-lactoglobulin allele is attenuated or rendered non-functional independently of inserting the lysozyme transgene. In these embodiments, an animal that has an attenuated or non-functional β-lactoglobulin allele is generated, and the lysozyme transgene is introduced into the animal with the attenuated or non-functional β-lactoglobulin allele. Alternatively, an animal that includes an attenuated or non-functional β-lactoglobulin allele is bred with a lysozyme transgenic animal, and the offspring have both a lysozyme transgene and an attenuated or non-functional β-lactoglobulin allele. Alternatively, one or more mutations are introduced into a β-lactoglobulin allele of a lysozyme transgenic animal.

A transgene that includes a coding region for lysozyme is used to transform a cell, meaning that a permanent or transient genetic change, generally a permanent genetic change, is induced in a cell following incorporation of the exogenous DNA of the transgene. A permanent genetic change is generally achieved by introduction of the DNA into the genome of the cell. Vectors for stable integration include plasmids, retroviruses and other animal viruses, BACs, HACs, YACs, and the like.

Transgenic animals of the invention comprise an exogenous nucleic acid sequence present as an extrachromosomal element or stably integrated in all or a portion of its cells, especially in germ cells. Unless otherwise indicated, it will be assumed that a transgenic animal comprises stable changes to the germline sequence. A subject transgenic animal may be heterozygous or homozygous for the transgene. During the initial construction of the animal, “chimeras” or “chimeric animals” are generated in some methods (e.g., where ES cells are used), in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

In some embodiments, the lysozyme transgene that is introduced into a cell includes an exogenous lysozyme coding sequence. The exogenous gene is in some embodiments from a different species than the animal host (e.g., is a heterologous lysozyme gene). The exogenous gene may or may not be altered in its coding sequence. Non-coding sequences, such as control elements, may or may not be present. Control elements, if present in the transgene, include homologous (e.g., normally associated with the coding sequence) or heterologous (e.g., not normally associated with the coding region, e.g., from another species). The introduced gene may be a wild-type gene, naturally occurring polymorphism, or a genetically manipulated sequence, for example having deletions, substitutions or insertions in the coding or non-coding regions. The lysozyme coding region may be operably linked to a promoter, which may be constitutive or inducible, and other regulatory sequences required for expression in the host animal. Alternatively, the lysozyme coding region may not be operably linked to a control element(s) in the transgene, but instead becomes operably linked to control element(s) when it becomes integrated into the genome. By “operably linked” is meant that a DNA sequence and a regulatory sequence(s) are connected in such a way as to permit gene expression when the appropriate molecules, e.g. transcriptional activator proteins, are bound to the regulatory sequence(s).

In other embodiments, the lysozyme transgene that is introduced into a cell includes an endogenous lysozyme coding sequence. In these embodiments, the coding sequence may or may not be operably linked to control element(s). The lysozyme coding region may be operably linked to a promoter, which may be constitutive or inducible, and other regulatory sequences required for expression in the host animal. Alternatively, the lysozyme coding region may not be operably linked to a control element(s) in the transgene, but instead becomes operably linked to control element(s) when it becomes integrated into the genome.

In some embodiments, a subject transgenic animal is produced by introducing into a single cell embryo a polynucleotide that comprises a nucleotide sequence that encodes lysozyme, or fragments or variants thereof, in a manner such that the polynucleotide is stably integrated into the DNA of germ line cells of the mature animal, and is inherited in normal Mendelian fashion. In accordance with the invention, a polynucleotide can be introduced into an embryo by a variety of means to produce transgenic animals. For instance, totipotent or pluripotent stem cells or somatic cells can be transformed by microinjection, calcium phosphate mediated precipitation, liposome fusion, retroviral infection or by other means. The transformed cells can then be introduced into embryos and incorporated therein to form transgenic animals.

In many embodiments, a polynucleotide is injected into an embryo, e.g., at the single-cell stage, forming a genetically modified embryo, and the genetically modified embryo is allowed to develop into a mature transgenic animal.

In some embodiments, the transgene is introduced into a somatic cell, where the transgene is integrated into the genome, forming a genetically modified somatic cell, and the nucleus of the genetically modified somatic cell is transferred into a single-cell embryo, forming a genetically modified embryo. The genetically modified single-cell embryo is then transferred into an oviduct of a recipient female, and the embryo allowed to develop into a mature transgenic animal.

Any method of making transgenic animals can be used as described, for example, in Transgenic Animal Generation and Use L. M. Houdebine, Harwood Academic Press, 1997; Transgenesis Techniques: Principles and Protocols D. Murphy and D. A. Carter, ed. (June 1993) Humana Press; Transgenic Animal Technology: A Laboratory Handbook C. A. Pinkert, ed. (January 1994) Academic Press; Transgenic Animals F. Grosveld and G Kollias, eds. (July 1992) Academic Press; and Embryonal Stem Cells: Introducing Planned Changes into the Animal Germline M. L. Hooper (January 1993) Gordon & Breach Science Pub; U.S. Pat. No. 6,344,596; U.S. Pat. No. 6,271,436; U.S. Pat. No. 6,218,596; and U.S. Pat. No. 6,204,431; Maga and Murray (1995) Bio/Technol. 13:1452-1457; Ebert et al. (1991) Bio/Technol. 9:835-838; Velander et al. (1992) Proc. Natl. Acad. Sci. USA 89:12003-12007; Wright et al. (1991) Bio/Technol. 9:830-834.

Transgenic animals also can be generated using methods of nuclear transfer or cloning using embryonic or adult cell lines as described for example in Campbell et al. (1996) Nature 380: 64-66; and Wilmut et al. (1997) Nature 385: 810-813. Cytoplasmic injection of DNA can be used, as described in U.S. Pat. No. 5,523,222. Subject transgenic animals can be obtained by introducing a chimeric construct comprising lysozyme-encoding sequences.

Transgenic animals also include somatic transgenic animals, e.g., transgenic animals that include a transgene in somatic cells (and not in germ line cells). For example, the mammary gland cells of an animal are transformed with a lysozyme transgene, and the transgene is expressed in mammary cells of the animal. Methods of somatic cell transformation are described in the art. See, e.g., Furth et al. (1995) Mol. Biotechnol. 4:121-127.

Methods for making transgenic goats are known in the art. See, e.g., Zou et al. (2002) Mol. Reprod. Dev. 61:164-172; Baldassare et al. (2002) Theriogenol. 57:275-284; and Ko et al. (2000) Transgenic Res. 9:215-222. Methods for making lysozyme transgenic goats are also described in the Examples. Methods for making transgenic cows are known in the art, and are described in, e.g., van Berkel et al. (2002) Nat. Biotechnol. 20:484-487.

Expression Vectors and Transgenes

A subject transgenic animal is typically generated by a method involving introducing into a cell a construct comprising a nucleotide sequence encoding lysozyme. A “lysozyme transgene” includes, at a minimum, a coding region for lysozyme. In some embodiments, the nucleotide sequence encoding lysozyme is operably linked to a promoter and, optionally, additional control elements, that provide for tissue-specific expression of the transgene in the animal. In other embodiments, the nucleotide sequence encoding lysozyme is not operably linked to any control elements. Instead, the lysozyme transgene includes, on the 5′ and 3′ ends of the coding region, sequences that provide for homologous recombination with an endogenous gene.

Any known coding sequence for lysozyme can be used to make a subject transgenic animal, including a lysozyme coding sequence from rat, mouse, human, cow, goat, sheep, etc. The coding sequence can be a cDNA sequence, or a genomic sequence. The coding sequence for the lysozyme may be, but need not be, from the same species as the transgenic animal. In many embodiments, the lysozyme transgene includes a coding region for human lysozyme.

The nucleotide sequences of mRNAs encoding lysozyme from a variety of animal species are known. Exemplary sequences are found under the following GenBank Accession numbers: mouse lysozyme mRNA, NM_{—}017372; rat lysozyme mRNA, NM_{—}012594 and L12458; human lysozyme mRNA, NM_{—}000239.

In addition, sequences that vary from a known coding sequence for lysozyme can be used, as long as the encoded lysozyme has substantially the same enzymatic activity. For example, the encoded lysozyme can include one or more conservative amino acid substitutions compared to the amino acid sequence of a known lysozyme. Examples of conservative amino acid substitutions are Phe/Tyr; Ala/Val; Leu/Ile; Arg/His; Ser/Thr; etc. The encoded lysozyme can also include insertions or deletions (including truncations) of one or more amino acid residues, compared to the amino acid sequence of a known lysozyme. Further, the encoded lysozyme can include one or more naturally occurring polymorphisms.

A suitable nucleotide sequence encoding a lysozyme generally has aa least about 75%, at least about 80%, at least about 85%, at least about 90%, at least about 95%, or at least about 98%, or higher, nucleotide sequence identity with a known coding sequence for lysozyme. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10 (using default settings).

Also suitable for use are lysozyme coding sequences that hybridize under stringent hybridization conditions to a known lysozyme coding sequence. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 1 ×SSC (150 mM NaCl, 15 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. For example, high stringency conditions include aqueous hybridization (e.g., free of formamide) in 6×SSC (where 20×SSC contains 3.0 M NaCl and 0.3 M sodium citrate), 1% sodium dodecyl sulfate (SDS) at 65° C. for about 8 hours (or more), followed by one or more washes in 0.2×SSC, 0.1% SDS at 65° C. For example, moderate stringency conditions include aqueous hybridization (e.g., free of formamide) in 6×SSC, 1% SDS at 65° C. for about 8 hours (or more), followed by one or more washes in 2×SSC, 0.1% SDS at room temperature.

As noted above, in some embodiments, a lysozyme transgene includes a coding sequence for lysozyme operably linked to one or more control sequences, e.g., promoters, 3′ transcriptional control sequences, translational control elements, etc. In some embodiments, a lysozyme transgene will include a control element that provides for increased mRNA stability.

In many embodiments, a lysozyme transgene includes a coding region for lysozyme operably linked to one or more tissue-specific control elements, e.g., a tissue-specific promoter, and optionally additional tissue-specific control elements (e.g., a 3′ untranslated region, an enhancer, and the like). The tissue-specific control element(s) can be heterologous, e.g., not normally operably linked to a lysozyme coding sequence in nature, or homologous, e.g., normally operably linked to a lysozyme coding sequence in nature. Tissue-specific control elements provide for expression of the lysozyme transgene preferentially in a given tissue, e.g., such control elements are more active (e.g., 2-fold, 5-fold, 10-fold, 20-fold, or 50-fold more active, or greater than 50-fold more active) in a given tissue than in other tissues under normal physiological conditions. A wide variety of tissue-specific promoters are known in the art.

Promoters useful for production of lysozyme in the milk of a subject transgenic animal are active in mammary tissue, e.g., the promoters are more active in mammary tissue than in other tissues under physiological conditions in which milk is synthesized. Suitable promoters provide for both specific and efficient transcription in mammary tissue. Mammary gland-specific promoters are strong promoters in mammary tissue that can support the synthesis of large amounts of protein for secretion into milk. Mammary gland-specific promoters include, but are not limited to, a whey acidic protein (WAP) promoter; αS1 casein, αS2 casein, β casein, and kappa casein promoters; an α-lactalbumin promoter; a lactoferrin promoter; and a α-lactoglobulin (“BLG”) promoter. The sequences of a number of mammary gland-specific promoters have been isolated and their nucleotide sequences have been published. See, for example, Clark et al. (1987) TIBTECH 5:20; and Henninghausen (1990) Protein Expression and Purification 41:3.

Where the control element operably linked to the lysozyme coding region in the transgene is a lysozyme control element, the lysozyme control element may be altered to provide for increased transcription, increased mRNA stability, and the like, e.g., using random or site-specific mutagenesis techniques. Methods for random and site-specific mutagenesis are well known in the art. Whether a given mutation of a control element increases the level of lysozyme mRNA is readily determined using well-known methods. For example, a an expression vector that includes a lysozyme promoter operably linked to a reporter gene, e.g., a nucleotide sequence encoding a detectable protein, such as a luciferase-encoding sequence, is introduced into a eukaryotic cell, and the promoter activity is determined by measuring the level of luciferase produced in the cell.

In some embodiments, a lysozyme transgene is not operably linked to a complete or functional control element. Instead, the transgene includes sequences that provide for homologous recombination with an endogenous gene, such that the lysozyme coding sequence replaces all or part of endogenous coding sequence, and the integrated lysozyme coding region is under transcriptional control of endogenous control element(s). For example, a lysozyme transgene includes 5′ and 3′ flanking sequences that are homologous to sequences in the 5′ and 3′ regions of a β-lactoglobulin gene, such that the transgene integrates into the genome of a cell by homologous recombination, whereby the lysozyme coding sequences of the transgene replace the endogenous β-lactoglobulin gene, and the lysozyme coding sequence integrates into the genome and is under the transcriptional control of the endogenous β-lactoglobulin control elements. Methods for carrying out homologous recombination are well known in the art.

A lysozyme transgene is generally provided as part of a vector (e.g., a lysozyme construct), a wide variety of which are known in the art and need not be elaborated upon herein. Vectors include, but are not limited to, plasmids; cosmids; viral vectors; artificial chromosomes (HACs, YACs, BACs, etc.); mini-chromosomes; and the like. Vectors are amply described in numerous publications well known to those in the art, including, e.g., Short Protocols in Molecular Biology, (1999) F. Ausubel, et al., eds., Wiley & Sons. Vectors may provide for expression of the subject nucleic acids, may provide for propagating the subject nucleic acids, or both.

For expression, e.g., where the transgene includes a promoter, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to a gene encoding the subject peptides, or may be derived from exogenous sources.

Where the transgene includes a promoter, an expression vector will generally have convenient restriction sites located near the promoter sequence to provide for the insertion of nucleic acid sequences encoding lysozyme. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region.

Fertilized Eggs

The present invention further provides isolated fertilized eggs derived from a subject female transgenic ungulate. The term “fertilized egg” includes a fertilized egg at any stage after fertilization and prior to implantation, e.g., from a fertilized one-cell egg to the blastocyst stage.

A subject isolated fertilized egg is useful for generating a subject transgenic ungulate. Thus, a subject isolated fertilized egg is capable of developing into a lysozyme transgenic animal having an attenuated or non-functional β-lactoglobulin allele when placed in the uterus of a recipient animal of the same species, e.g., a pseudopregnant recipient female animal of the same species. The term “isolated,” in the context of a fertilized egg, refers to a fertilized egg that has been removed from a subject transgenic ungulate.

A subject isolated fertilized egg may be obtained by flushing of the uterus after fertilization and retrieval of fertilized eggs. A subject isolated fertilized egg may be stored for an extended period of time, or used immediately.

Where the subject isolated fertilized egg is stored, the fertilized egg is stored in a frozen, refrigerated, or vitrified state. Where the isolated fertilized egg is frozen, a suitable cryoprotectant compound is typically added to the fertilized egg or the medium containing the fertilized egg. Suitable cryoprotective compounds include permeating and nonpermeating compounds. Most commonly, dimethyl sulfoxide (DMSO), glycerol, propylene glycol, ethylene glycol, or the like are used as permeating cryoprotective agents. Other permeating agents include propanediol, dimethylformamide and acetamide. Nonpermeating agents include polyvinyl alcohol, polyvinyl pyrrolidine, anti-freeze fish or plant proteins, carboxymethylcellulose, serum albumin, hydroxyethyl starch, Ficoll, dextran, gelatin, albumin, egg yolk, milk products, lipid vesicles, or lecithin. Adjunct compounds that may be added include sugar alcohols, simple sugars (e.g., sucrose, raffinose, trehalose, galactose, and lactose), glycosaminoglycans (e.g., heparin, chrondroitin sulfate), butylated hydroxy toluene, detergents, free-radical scavengers, and anti-oxidants (e.g., vitamin E, taurine), and amino acids (e.g., glycine, glutamic acid).

Following suspension of the cells in the cryoprotective medium (e.g., for storage), the container is sealed and subsequently either refrigerated or frozen. Briefly, for refrigeration, the sample is placed in a refrigerator in a container filled with water for one hour or until the temperature reaches 4° C. If the sample is to be frozen, the cold sample is aliquoted into cryovials or straws and placed in the vapor phase of liquid nitrogen for one to two hours, and then plunged into the liquid phase of liquid nitrogen for long-term storage or frozen in a programmable computerized freezer. Frozen samples are thawed by warming in a 37° C. water bath and are directly used, or washed, and then used. Other cooling and freezing protocols may be used. Vitrification involves dehydration of the fertilized egg using sugars, Ficoll, or the like. The oocyte or embryo is then added to a cryoprotectant and rapidly moved into liquid nitrogen. Refrigeration is generally an appropriate means for short-term storage, while freezing or vitrification are generally appropriate means for long or short-term storage.

Utility

The subject transgenic animals find use in a variety of applications, including, but not limited to, milk production, processed milk product production, research, and the like. For example, the subject animals find use in producing milk that has decreased rennet clotting time and that results in increased efficiency of cheese production. The subject animals find use in research, to analyze the effects of lysozyme levels on production of milk and milk-based products. Because milk from subject female transgenic animals have lower levels of β-lactoglobulin, the milk from the animals is particularly suitable for infant formula and processing.

Food Applications

The present invention provides methods for producing milk from a subject transgenic animal, as well as methods for producing milk-based food products from milk harvested from a subject transgenic animal. The methods generally involve harvesting milk from a subject transgenic animal. Where the food product requires further processing, the methods involve harvesting milk from a subject transgenic animal; and processing the food product.

The invention further provides milk produced by a subject transgenic ungulate, as well as food products made from milk harvested from a subject transgenic ungulate. A subject food product made from milk harvested from a transgenic ungulate includes a food product that contains a milk of a subject transgenic ungulate, or a product produced from the milk of a subject transgenic animal. Food products include any preparation for human consumption including for enteral or parenteral consumption, which when taken into the body (a) serve to nourish or build up tissues or supply energy and/or (b) maintain, restore or support adequate nutritional status or metabolic function. Food products of the invention are suitable for human consumption.

A subject food product includes milk, and any food products made from or containing milk, including, but not limited to, cheese, yogurt, butter, ice cream, and other frozen desserts, whipped toppings, cream, custard, pudding, nutritional drinks, infant formula, and chocolate.

Milk produced by a subject transgenic ungulate has a level of enzymatically active lysozyme that is at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 4-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, at least about 1200-fold, at least about 1500-fold, at least about 1700-fold, at least about 2000-fold, at least about 2500-fold, or at least about 3000-fold, or more, higher than the level of enzymatically active lysozyme in the milk of a female non-transgenic animal of the same species. For example, the level of enzymatically active lysozyme in the milk of a subject transgenic ungulate is from about 0.10 mg/ml to about 2.0 mg/ml, from about 0.2 mg/ml to about 1.0 mg/ml, or from about 0.4 mg/ml to about 0.8 mg/ml. In some embodiments, the level of enzymatically active lysozyme in the milk of a subject transgenic ungulate is greater than 2.0 mg/ml, e.g., from about 2.0 mg/ml to about 7.5 mg/ml, e.g., from about 2.0 mg/ml to about 2.5 mg/ml, from about 2.5 mg/ml to about 3.0 mg/ml, from about 3.0 mg/ml to about 3.5 mg/ml, from about 3.5 mg/ml to about 4.0 mg/ml, from about 4.0 mg/ml to about 5.0 mg/ml, from about 5.0 mg/ml to about 6.0 mg/ml, from about 6.0 mg/ml to about 7.0 mg/ml, or from about 7.0 mg/ml to about 7.5 mg/ml.

Milk produced by a subject transgenic ungulate is at least about 5%, at least about 10%, at least about 15%, at least about 20%, at least about 25%, at least about 30%, at least about 35%, at least about 40%, at least about 45%, or at least about 50%, at least about 60%, at least about 70%, at least about 80%, at least about 90%, at least about 2-fold, at least about 4-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, at least about 1200-fold, at least about 1500-fold, at least about 1700-fold, at least about 2000-fold, at least about 2500-fold, or at least about 3000-fold, or more, bacteriostatic than milk from a non-transgenic ungulate of the same species. In particular, the milk of a subject ungulate exhibits bacteriostatic activity toward mastitis-causing bacteria, and toward bacteria that cause food spoilage. Such bacteria include, but are not limited to, Escherichia coli; various staphylococcal species, including, e.g., Staphylococcus areus; Pseudomonas species, including, e.g., Pseudomonas fragi, P. fluorescens, P. aeruginosa; streptococcal species, including e.g., Streptococcus cremoris, S. agalactiae, S. uberis; Lactococcus species, including, e.g., L. lactis; Lactobacillus species; and the like.

The milk, or a product made from milk of a subject transgenic ungulate has increased shelf life compared to the shelf life of the same milk product made from milk of a non-transgenic animal of the same species. For example, the shelf life of milk or a product made from the milk of a subject transgenic ungulate has a shelf life that is at least about 10%, at least about 15%, at least about 20%, at least about 25%, at least about 30%, at least about 35%, at least about 40%, at least about 50%, at least about 60%, at least about 70%, at least about 80%, at least about 90%, at least about 2-fold, at least about 3-fold, at least about 4-fold, or at least about 5-fold or more greater than the shelf life of the milk or the same milk product made from milk of a non-transgenic animal of the same species.

The milk of a subject transgenic ungulate has a level of μ-lactoglobulin that is at least about 5%, at least about 10%, at least about 15%, at least about 20%, at least about 25%, at least about 30%, at least about 35%, at least about 40%, at least about 45%, or at least about 50%, at least about 60%, at least about 70%, at least about 80%, or at least about 90% less than the level of β-lactoglobulin in the milk of a non-transgenic animal of the same species.

The milk of a subject transgenic ungulate has reduced rennet clotting time, compared to the milk of a non-transgenic ungulate of the same species. The rennet clotting time is the time required for curd formation to occur. A reduction in rennet clotting time provides for enhanced cheese production. The rennet clotting time of milk of a subject transgenic ungulate is reduced by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90%, or more, compared to the rennet clotting time of milk of a non-transgenic ungulate of the same species.

Cheese produced from the milk of a subject transgenic ungulate has increased gel strength compared to cheese made by the same process from milk of a non-transgenic ungulate of the same species. The gel strength of cheese produced from the milk of a subject transgenic ungulate is at least about 5%, at least about 10%, at least about 15%, at least about 20%, at least about 25%, at least about 30%, at least about 35%, at least about 40%, at least about 45%, at least about 50%, or more, greater than the gel strength of cheese made by the same process from milk of a non-transgenic ungulate of the same species.

Methods of Making Cheese

Methods of making cheese are known in the art, and a subject food product harvested from a subject ungulate can be used in the production of cheese using any known method. Rennet is added to milk from a subject transgenic ungulate, using standard industry methods. One or more additional curd formation-inducing agents can be added. Curd formation inducing agents include polycations other than lysozyme, including, but not limited to, salmine and calcium.

In general, a subject method of making cheese involves adding rennet or another curd formation inducing agent to milk from a subject transgenic ungulate; allowing curd formation to occur; and producing cheese from the curds. In some embodiments, the method involves adjusting the pH of the milk harvested from a subject transgenic animal; adding one or more curd formation inducing agents; allowing curd formation to occur; separating curd from whey; and forming the curd into a cheese product. The process may further include a curing step.

Typically, rennet is added in an amount of from about 1:1000 to about 1:15000 rennet to milk, e.g., from about 1:1000 to about 1:2000, from about 1:2000 to about 1:4000, from about 1:4000 to about 1:6000, from about 1:6000 to about 1:8000, from about 1:8000 to about 1:10,000, or from about 1:10,000 to about 1:15,000, although ratios of rennet:milk that are lower or higher can also be used. Curd formation is allowed to proceed at a temperature of from about 32° C. to about 35° C., although higher or lower temperatures can also be used, e.g. curd formation can be allowed to proceed at a temperature of from about 25° C. to about 32° C., or from about 35° C. to about 42° C. Curd formation is allowed to proceed for a period of time of from about 5 minutes to about 30 minutes, although shorter or longer curd formation times can be used, e.g., curd formation can be allowed to proceed for a period of from about 1 minute to about 60 minutes.

Cheeses that can be made using a method of the invention include any fresh, or ripened cheese that are made by a process that includes curd formation. Such cheese include, but are not limited to, Campesino, Chester, Danbo, Drabant, Herregard, Manchego, Provolone, Saint Paulin, Soft cheese, Taleggio, White cheese, Cheddar, Colby, Edam, Muenster, Gruyere, Emmenthal, Camembert, Parmesan, Romano, Mozzarella, Feta; cream cheese, Neufchatel, etc.

In some embodiments, the method further involves processing the cheese into a processed cheese food product. Processed cheese food products include, but are not limited to, pizza, ready-to-eat dishes, toast, burgers, lasagna, dressing, sauces, cheese powder, cheese flavor, and processed cheese.

Research Applications

The subject transgenic animals find use in research, to analyze the effects of increased levels of lysozyme and decreased β-lactoglobulin levels on milk quality. In addition, the subject transgenic animals are useful for studying the regulation of transcription and translation of a lysozyme gene.

For example, mutations may be introduced into the lysozyme promoter region to determine the effect of altering expression in a lysozyme transgenic animal.

Lysozyme regulatory sequences incorporated into a transgene may be used to identify cis acting sequences required for transcriptional or translational regulation of expression, especially in different tissues or stages of development, and to identify cis acting sequences and trans-acting factors that regulate or mediate expression. Such transcription or translational control regions may be operably linked to a reporter gene or a lysozyme gene to examine the effects of the regulatory sequences on expression levels and mRNA stability.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Standard abbreviations may be used, e.g., bp, base pair(s); kb, kilobase(s); pl, picoliter(s); s, second(s); min, minute(s); hr, hour(s); and the like.

### Example 1

### Generating Transgenic Animals

Injection Material

All injection materials (probes) were designed using the goat β-lactoglobulin (β-lg) gene sequences in combination with the cDNA for human lysozyme inserted in frame with the β-lg start codon. A 311 base pair (from −157 to +154) DNA fragment of the goat β-lactoglobulin (β-lg) gene was amplified from goat genomic DNA by polymerase chain reaction (PCR) with primers A (5′-AAATGGTACCGGGGCCCGGGGATGAGCCAA-3′; SEQ ID NO:01) and B (5′-AAATTCTAGATGAGGCCCAGCTCCCCTGCC-3′; SEQ ID NO:02) and cloned into pBluscript SK (Stratagene, La Jolla, Calif.) by the use of KpnI and XbaI sites included in the primers. The resulting plasmid (PBLG1) was then modified by PCR to replace the translation start codon in exon 1 with a 13 bp insertion sequence (5′-GCGGCCGCTCGAG-3′; SEQ ID NO:05) containing the unique restriction enzyme sites XhoI and NotI with primers A and F (5′-GCGGCCGCTCGAGGGCTGCAGCTGGGGTCGTG-3′; SEQ ID NO:03) as well as B and E (5′-CTCGAGCGGCCGCAAGTGCCTCCTGCTTGCCCT-3′; SEQ ID NO:04) for the first few cycles followed by amplification of the modified fragment with primers A and B. The resulting plasmid (pBlg-KO) contained 311 bp of goat β-lg DNA surrounding the start codon that was replaced with the 13 bp insertion sequence. The introduction of the 13 bp insertion sequence also generated a 3 bp deletion, resulting in a frame shift mutation in exon 1 of the goat β-lg gene. Probe GHLZ had the complete 540 bp cDNA for human lysozyme (Maga et al. (1994) Transgenic Res. 3:36-42) inserted into the XhoI site in the plasmid pBlgKO. The orientation of the human lysozyme insert was verified by restriction enzyme digests and DNA sequencing.

Preparation of Injection DNA

The DNA to be injected was removed from vector sequences with appropriate restriction enzymes and purified with Elutip-D columns (Schleicher & Schuell, Keene, NH) or by polymerase chain reaction (PCR) with primers A and B prior to microinjection. For the RecA protein coating of the DNA, linear, double-stranded DNA (200 ng) was heat denatured at 98° C. for 5 min, cooled on ice for 1 min and added to a protein coating mix containing tris-acetate buffer, 20 mM magnesium acetate and 0.2-2.4 mM ATPγS. RecA protein (Roche, Indianapolis, Ind.) was immediately added and the reaction incubated at 37° C. for 15 min and the magnesium acetate concentration was increased to a final concentration of 11 mM. The RecA protein coating of the DNA was monitored by agarose gel electrophoresis with uncoated double-stranded DNA as control. The electrophoretic mobility of RecA protein-coated DNA was significantly retarded as compared with non-coated double stranded DNA. RecA-protein coated DNA was diluted to a concentration of 5 ng/μl with water and used for the standard pronuclear microinjection of one-cell goat zygotes.

Generation of Embryos

Pronuclear stage goat embryos were obtained from donor animals in which estrus was synchronized by using progestin pessaries (Redopharm, Ltd.) for 14 days. On day 13, follicle stimulating hormone (FSH) was given twice daily (intramuscular; IM) over 3 days, beginning with a dose of 5 mg the first day, 4 mg the next day and 3 mg the third day if needed, with removal of the progestin sponge on day 14. Twenty-four hours after progestin removal, gonadotropin releasing hormone (GnRH Cystorelin^{R}, Walco Int'l.) was administered (5 mg dose, intravenous; IV) to all animals, and does in estrus were bred to fertile bucks. Embryos were recovered by oviductal flushes on day 2, where day 0 is the first day of estrus. Estrus in recipient females was synchronized to correspond with the donors by use of progestin pessaries for 14 days. Microinjected embryos were surgically transferred into the oviducts of recipient does via midline laparotomy on the same day. Pregnancies were confirmed and monitored by ultrasound at days 28, 35, 47 and 54 following embryo transfer.

Analysis of Animals

Samples of umbilical cord were taken at birth and ear notch were obtained within one week of age from all offspring. DNA was prepared from tissue samples by incubation in digestion buffer (0.05 M Tris, 0.1M EDTA, 10% SDS, 20 mg/ml Proteinase K) at 55° C. overnight followed by phenol chloroform extraction. Transgenic animals were initially identified by polymerase chain reaction (PCR) analysis. A PCR was first performed with an endogenous set of primers to serve as an internal PCR control. The internal control amplified an endogenous 369 bp region spanning the start codon in exon 1 of the goat β-lg gene.

To identify transgenic animals, PCR was performed in triplicate on each tissue sample with primer sets within the injected DNA sequence. For probe GHLZ a 243 bp product specific to the human lysozyme cDNA was amplified with primers HL3/HL4 if the animal was transgenic. These primers spanned exons in the human lysozyme cDNA. For all PCR reactions, a total of 0.1 μg of genomic DNA from potential transgenic offspring was added to a standard PCR reaction containing buffer, 1.5 mM MgCl₂, 10 mM dNTP's, 10 pmol each of primer and 2.5 Units of Taq DNA polymerase in a final volume of 50μl. Samples were subjected to a single denaturation step of 97° C. for 2min followed by 30-35 cycles of 94° C. for 1 min, annealing at 58° C. for 1 min and extension at 7° C. for 1 min. Products were analyzed by standard ethidium bromide agarose gel electrophoresis. PCR products from all animals scoring positive for integrated DNA were sequenced to verify the identity of the PCR product. Multiple PCR primer sets were run for each line of animals.

Southern blots were performed on all PCR-positive animals as well as negative control animals to verify further the presence of the injected DNA. Briefly, 15 μg of genomic DNA was digested with TaqI overnight and run on a 1% gel overnight at 35V and transferred to a nylon membrane (Hybond+, Amersham Pharmacia) in 0.4N NaOH. Membranes were probed with the human lysozyme cDNA labeled by random priming with α³²P-dCTP. Hybridization and standard washes (0.1% SSC/0.1% SDS as final wash) were carried out at 65° C.

### Example 2

### Characterization of Milk from Transgenic Goats

Milk was collected from lactating lysozyme transgenic goats (Example 1). Milk was collected, stored at room temperature for the indicated lengths of time, then an aliquot of milk was plated on an agar plate containing a general bacterial growth medium. Plates were incubated at 37° C. overnight. Typical results of shelf life studies with lysozyme transgenic and non-transgenic goat milk are shown in Table 1.

TABLE 1

| Day | Transgenic | Control |
|---|---|---|
| 0 | 0 (0) | 130 (26%) |
| 1 | 3,677 (27%) | 11,385 (56%) |
| 2 | 10,400 (40%) | 24,824 (74%) |

Milk was collected from each half of the udder and combined and plated at collection (Day 0) and after 24 (Day 1) and 48 (Day 2) hours at room temperature. Values are the mean colony forming units (CFU)/ml formed on plates for 6 trials each using milk from 3 lysozyme transgenic and 5 non-transgenic control goats. Numbers in parentheses are the proportion of milk samples culturing positive for bacterial growth.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A transgenic ungulate comprising a transgene encoding lysozyme, wherein said transgenic ungulate comprises a non-functional β-lactoglobulin allele, wherein said ungulate is female, and wherein said lysozyme-encoding transgene is expressed in mammary gland cells of said ungulate, wherein milk produced by said transgenic ungulate has a level of lysozyme that is at least about 10% higher than the level of lysozyme in milk of a non-transgenic ungulate of the same species, and wherein the milk has a level of β-lactoglobulin that is at least about 10% lower that the level of β-lactoglobulin in milk of a non-transgenic ungulate of the same species.

2. The transgenic ungulate of claim 1, wherein said transgene comprises a coding sequence for lysozyme operably linked to a mammary specific promoter.

3. The transgenic ungulate of claim 1, wherein said ungulate is a cow.

4. The transgenic ungulate of claim 1, wherein said ungulate is a goat.

5. The transgenic ungulate of claim 1, wherein said transgene is chromosomally integrated.

6. The transgenic ungulate of claim 1, wherein said ungulate is heterozygous for the transgene and heterozygous for the non-functional β-lactoglobulin allele.

7. The transgenic ungulate of claim 1, wherein said ungulate is homozygous for the transgene and homozygous for the non-functional β-lactoglobulin allele.

8. The transgenic ungulate of claim 1, wherein the transgene encodes human lysozyme.

9. An isolated fertilized egg, wherein said egg is isolated from a transgenic ungulate comprising a transgene encoding lysozyme and comprising a non-functional β-lactoglobulin allele.

10. A composition comprising an isolated fertilized egg according to claim 9; and a cryoprotective agent.

11. A method of producing a food product, said method comprising harvesting a food product from a transgenic ungulate of claim 1.

12. A method of producing a food product, the method comprising processing a food product harvested from a transgenic ungulate of claim 1.

13. A method of producing cheese, the method comprising:
a) adding rennet to milk harvested from a transgenic ungulate according to claim 1;
b) allowing curd formation to occur; and
c) producing cheese from the curds.

14. A milk product produced by a transgenic ungulate of claim 1, wherein said milk product has a level of lysozyme that is at least about 10% higher than the level of lysozyme in milk of a non-transgenic ungulate of the same species, and wherein said milk product has a level of β-lactoglobulin that is at least about 10% lower than the level of β-lactoglobulin in milk of a non-transgenic ungulate of the same species.

15. The milk product of claim 14, wherein the milk product has reduced rennet clotting time compared to a milk product of a non-transgenic animal of the same species.

16. A processed milk produced from milk of a transgenic ungulate of claim 1.

17. The processed milk product of claim 16, wherein the processed milk product is cheese.

18. The processed milk product of claim 17, wherein the cheese has increased gel strength compared to cheese made from milk of a non-transgenic ungulate of the same species.


SEQUENCE LISTING (5 sequences)

SEQ ID NO: 1 - DNA, 30 bp, Artificial Sequence
  primer
aaatggtaccggggcccggggatgagccaa

SEQ ID NO: 2 - DNA, 30 bp, Artificial Sequence
  primer
aaattctagatgaggcccagctcccctgcc

SEQ ID NO: 3 - DNA, 32 bp, Artificial Sequence
  primer
gcggccgctcgagggctgcagctggggtcgtg

SEQ ID NO: 4 - DNA, 33 bp, Artificial Sequence
  primer
ctcgagcggccgcaagtgcctcctgcttgccct

SEQ ID NO: 5 - DNA, 13 bp, Artificial Sequence
  synthetic DNA
gcggccgctcgag